(19) United States
(12) Patent Application Publication
(10) Pub. No.: US 2024/0216531 A1
(43) Pub. Date: Jul. 4, 2024

(54) CHITOSAN-CONTAINING NANOPARTICLES FOR DELIVERY OF POLYNUCLEOTIDES

(71) Applicants: VALORISATION RECHERCHE HSCM, LIMITED PARTNERSHIP, Québec (CA); UNESP UNIVERSIDADE ESTADUAL PAULISTA "JULIO DE MESQUITA FILHO" (STATE UNIVERSITY), Sao Paulo, SP (BR)

(72) Inventors: Marcio José TIERA, Sao José do Rio Preto (BR); Julio Cesar FERNANDES, Montréal (CA); André Miguel MARTINEZ JUNIOR, Cedral-SP (BR); Ricchard Hallan Felix VIEGAS DE SOUZA, Sao José do Rio Preto, SP (BR); Mohamed BENDERDOUR, Pierrefonds (CA); Francis VALLIERES, Laval (CA)

(21) Appl. No.: 18/552,321

(22) PCT Filed: Mar. 25, 2022
(86) PCT No.: PCT/CA2022/050448

§ 371 Date: Sep. 25, 2023

Related U.S. Application Data
(60) Provisional application No. 63/166,310, filed on Mar. 26, 2021.

(51) Int. Cl. A61K 48/00; A61K 9/51; A61K 47/54; A61K 47/68; C12N 15/113

(52) U.S. Cl. CPC A61K 48/0041; A61K 9/5123; A61K 9/5146; A61K 9/5161; A61K 47/549; A61K 47/6835; C12N 15/113; C12N 2310/14

(57) ABSTRACT

Disclosed is a chitosan-containing vector as set forth in formula (I): wherein R₁, R₂, R₃, R₄, R₅, and R₆ are as defined herein, and w, p, and z are integers varying from 0 to 1500, the sum of which is between 50 and 1500, so as to define a chitosan backbone with a molecular weight between 10 kDa and 300 kDa. The chitosan-containing vector comprising 5% to 55% of N,N-diisopropylethylamine, relatively to the sum of w, p, and z, and up 5% for pegylated units. There is also described the use of this vector with a siRNA as a mean for transfection or transformation of the siRNA for treating various diseases.

### TECHNICAL FIELD

The present invention generally relates to a non-viral chitosan-based drug delivery systems.

### BACKGROUND OF THE ART

Rheumatoid arthritis (RA) is a chronic autoimmune disease with systemic inflammation characterized by pain, hookworm, swelling and destruction of joints (especially cartilage), as well as weakening of bone structures.

Patients are more likely to suffer from frailty fractures resulting from secondary osteoporosis and bone loss, which often results in a disability with a great economic impact on the health system.

The alpha tumor necrosis factor (TNF-α) is considered a major player in the pathophysiology of rheumatoid arthritis, especially in joint inflammation, cartilage and bone resorption. For this reason, TNF-α was chosen as a target, since production inhibition improves the inflammation conditions of arthritis, as indicated by treatments that target this cytokine using a monoclonal anti-TNF-α antibody.

Other targets are also being studied to control arthritic inflammation, such as the EP4 receptor, C5 and C5aR1 components of the supplement system, double specificity phosphatase 2, among others.

The interference RNA molecule (siRNA) is a small sequence of 19-21 nucleotides and is used as an innovative therapeutic procedure for the treatment of various diseases. However, in addition to being negatively charged with a limitation of its internalization by cells, in addition, it presents a strong homology with nonspecific targets, also being very sensitive to degradation by ribonucleases.

Therefore, an effective administration system is needed for encapsulation, transmission and protection against degradation by nucleases, so that the cells of interest are targeted. Therefore, for the release of siRNA into the cytoplasm of target cells, various viral and non-viral vectors are being studied.

In recent years, a variety of non-viral vectors have been proposed, including structural modifications of siRNA, liposomes, cationic polymers, peptides and site-directed systems.

There are numerous DNA/RNA delivery vehicles for treating various diseases through gene therapy. Lately Chitosan used for delivering siRNA was reported in various patent applications. Chitosan and its derivatives have been used as in vitro and in vivo delivery systems, mainly because of its properties as plasmid DNA release systems. Chitosan is widely used as biodegradable biomaterials with low toxicity and immunogenicity.

For this reason, in order to improve their effectiveness as a gene carrier and as a transfection agent, chitosan has been modified targeting its use as a transfection agent.

However, one of the limitations of chitosan is its low solubility at physiological pH.

Patent application HK1199215 (A1) describes a composition and process for the transport and release of RNA from interference in in vitro cells and in vivo is described using specific formulations of a non-viral distribution with chitosan without further modifications.

Similarly, Chinese applications CN103893753, CN103893754, CN103893755, CN103893756, and CN103893757 describe the use of chitosan to release a recombinant plasmid.

Additionally, Chinese application CN102908315 used chitosan, without further modification, for the transport and release of interference RNA.

Still applications US2009/0324726 and BR102016030231-5 A2 have reported a chitosan-based delivery vehicle.

However, the chitosan-based delivery vehicle described in the prior art is either toxic to cells or unstable, such that their commercialisation is close to impractical. Accordingly, it is desirable to design new chitosan-based delivery system that are stable and not cytotoxic.

### SUMMARY

In accordance with one aspect, there is provided a new chitosan-based nanoparticle for drug delivery that is stable, not cytotoxic and further is degradable.

In accordance with another aspect, there is provided a chitosan-containing vector as set forth in the following formula:

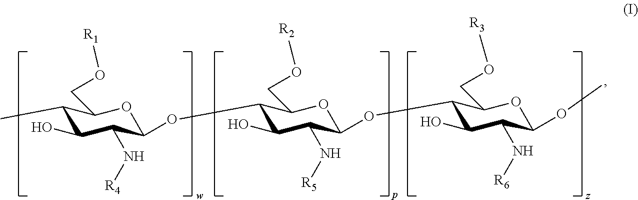

wherein
R₁, R₂, and R₃ are each independently hydrogen or

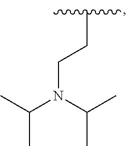 [SMILES: *CCN(C(C)C)C(C)C]

where [symbol] represents a point of attachment,
R₄, R₅ and R₆ are each independently hydrogen, —C(O)—CH₃, —C(O)CH₂—(O—CH₂—CH₂)ₙ—R₇, —C(O)CH₂—CH₂—R₈, or

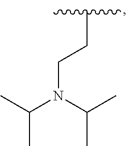 [SMILES: *CCN(C(C)C)C(C)C]

R₇ is hydrogen, hydroxyl, a ligand or a targeting moiety
R₈=is —S—S—R₉
R₉ is a (C₈-C₁₂)Alkyl or —CH₂—CH₂—(O—CH₂—CH₂)ₙ—OR₁₀,
R10 is hydrogen, a ligand or a targeting moiety,
n is an integer between 0 and 50,
w, p, and z are integers varying from 0 to 1500, the sum of w, p, and z being between 50 and 1500, so as to define a chitosan backbone with a molecular weight between 10 kDa and 300 kDa, said chitosan-containing vector comprising 5% to 55% of

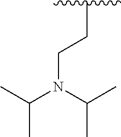 [SMILES: *CCN(C(C)C)C(C)C]

relatively to the sum of w, p, and z, and up 5% for pegylated units.

In a further aspect of the invention, there is provided a chitosan-containing vector comprising a backbone comprising diisopropylethylamine (DIPEA) covalently linked to the 6-hydroxyl group of chitosan or to the amino group on the C2 position of Chitosan.

The numbering of the atoms on the building block of chitosan is as follows:

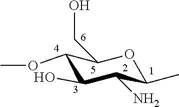

In some embodiments, the chitosan in the chitosan-containing vector has a molecular weight comprised between 10 kDa and 300 kDa, with a degree of deacetylation varying from 70% to 99%.

In some embodiments, the hydrogen of the hydroxy group on C6 or of the amine group on C2 is substituted between 5 and 55% with DIPEA.

In some embodiments, the backbone in the chitosan-containing vector further comprises polyethylene glycol (PEG) covalently attached directly or through a disulfide bridge to the amino group at position C2 of the chitosan. The PEG in some embodiments can have a molecular weight varying between 2 kDa and 10 kDa.

In some embodiments, the hydrogen of the amine group on C2 is substituted between 2 and 5% with PEG.

In other embodiments, in the chitosan-containing vector, the backbone further comprises a ligand or a targeting moiety. The ligand or a targeting moiety can be for example, without limitation, folic acid, aptamers, proteins, peptides, chimeric antibodies, humanized antibodies or monoclonal antibodies.

In accordance with the present invention, there is also provided a nanoparticle comprising the chitosan-containing vector as defined herein and a polynucleotide. The polynucleotide can be for example, without limitation, small interference RNA (siRNA), messenger RNA (mRNA) or DNA.

Still in accordance with the present invention, there is also provided a method for preparing a chitosan-containing vector, said method comprising the steps of:
reacting chitosan (Ch) with 2-Chloro-N,N-diisopropylethylamine hydrochloride (DIPEA-CI) to attach a N,N-diisopropylethylamine (DIPEA) to chitosan to obtain a N,N-diisopropylethylamine-chitosan (DIPEA-Ch); and
optionally reacting polyethylene glycol with N-Succinimidyl 3-(2-pyridyldithio) propionate (SPDP) to obtain PEG-SH and reacting said PEG-SH obtained with said DIPEA-Ch to obtain DIPEA-Ch-PEG.

The method as described herein may also comprises the step of purifying said DIPEA-ch or said DIPEA-Ch-PEG and may also preferably further comprise isolating said DIPEA-ch or said DIPEA-Ch-PEG. When purified, said DIPEA-ch or said DIPEA-Ch-PEG can be purified by dialysis.

In accordance with the present invention, the chitosan-containing vector as described herein can be used for transfection or transformation of a host cell.

In accordance with an aspect of the present invention, there is provided the use of the chitosan-containing vector as described herein, in a non-viral gene therapy.

In a further aspect of the invention, there is also provided the use of the chitosan-containing vector as described herein, for inhibiting or stimulating gene expression or protein translation in a host cell.

In a still further aspect of the invention, there is provided the use of the chitosan-containing vector as described herein, in the preparation of a drug for the treatment of a genetic disease, which is hereditary or acquired, preferably by gene therapy treatments, such as chronic inflammatory diseases mediated by TNF-α or other cytokine, Crohn's disease, ulcerative colitis, psoriasis and ankylosing spondylarthritis. Such drug can be for example intended for the treatment of an arthritic or inflammatory disease.

In another aspect of the invention, there is provided the use of the nanoparticle as described herein, for inhibiting or stimulating gene expression or protein translation in a host cell.

In a further aspect of the invention, there is also provided the use of the nanoparticle as described herein, in the preparation of drugs for the treatment of a genetic disease, which is hereditary or acquired, preferably by a gene therapy treatment. Such genetic disease can be for example chronic inflammatory diseases mediated by TNF-α or other cytokine, Crohn's disease, ulcerative colitis, psoriasis and ankylosing spondylarthritis, among others.

Many further features and combinations thereof concerning the present improvements will appear to those skilled in the art following a reading of the instant disclosure.

### DESCRIPTION OF THE DRAWINGS

FIGS. 1A and 1B illustrate the nuclear magnetic resonance spectra of hydrogen (NMR 1H) of medium molecular weight chitosan (CM) and its derivatives that contain increasing proportions of diisopropylethylamine (FIG. 1A) and increasing Poly (ethylene-glycol) and/or hydrophobic groups linked to chitosan chain via disulfide linkages (FIG. 1B).

FIGS. 2A and 2B illustrate the nuclear magnetic resonance spectra of hydrogen (NMR 1H) of high molecular weight chitosan (CH) and its derivatives that contain diisopropylethylamine (FIG. 2A) and Poly (ethylene glycol) (FIG. 2B).

FIGS. 3A and 3B illustrate the nuclear magnetic resonance spectra of chitosan (RMN 13C) of medium molecular weight (FIG. 3A) and high molecular weight (FIG. 3B) chitosan and its DIPEA-nuclear weight derivatives.

FIGS. 4A-4H illustrate the GPC chromatograms of CH (FIG. 4A), CHD (FIG. 4B), CHD32 (FIG. 4C), CM (FIG. 4D), CMD5 (FIG. 4E), CMD15 (FIG. 4F), CMD34 (FIG. 4G) and CMD55 (FIG. 4H) with the pullulan analytical curve.

FIG. 5 illustrates the formation of the nanoparticles obtained from the interaction of the chitosan derivatives modified with DIPEA and PEG with siRNA.

FIGS. 6A and 6B illustrates the titration curves of chitosan and DIPEA-Ch derivative.

FIGS. 7A and 7B graphically represent the variation in the degree of ionization of high molecular weight chitosan (7A) and medium molecular weight chitosan (7B) in relation to blood pH variation (pH 7.4) and in the early endosome (pH 6.3). FIGS. 7A-7B inserts are photographs of polymeric solutions (2 g L⁻¹) in phosphate buffer pH 7.4 with ionic strength adjusted in 150 mmole L⁻¹.

FIGS. 8A-80 represent images of electrophoreses (agarose gel) polyplexes prepared in increasing N/P reasons, at pH 7.4 and ion strength of 150 mmoL⁻¹. The arrows (rectangles) indicate the siRNA released by the nanovectors (nanoparticles).

FIGS. 9A-9F graphically illustrate the hydrodynamic diameter (9A and 9B), the potential zeta (9C and 9D) and the polydispersity index (9E and 9F) of polyplexes formed by the interaction of DIPEA-derived chitosan average molecular weight with siRNA, in increasing N/P ratios and under physiological conditions of pH (7.4) and ionic strength (150 mmoL⁻¹).

FIGS. 10A-10F graphically illustrate the hydrodynamic diameter (10A and 10B), the zeta potential (10C and 10D) and the polydispersivity index (10E and 10F) of polyplexes formed by the interaction of DIPEA-derived chitosan of high molecular weight with siRNA, in increasing N/P ratios and under physiological pH (7.4) and ionic strength conditions (150 mmole L⁻¹).

FIGS. 11A and 11B represent photographs of electronic scanning microscopy of the FEG type (MEV-FEG) of siRNA loaded nanoparticles formulated with CHD32 (11A) or CMD55-P1.3 (11B) at N/P ratio 10, in pH 7.4 and ionic strength of 150 mmole L⁻¹.

FIGS. 12A to 12D graphically illustrate the hydrodynamic diameter (12B and 12D) and the polydispersivity index (12A and 12C) of nanoparticles formed by medium (12A and 12B) and high (12C and 12D) molecular weight polymers over time. All nanoparticles were prepared in phosphate buffer at N/P ratio 10, ionic strength of 150 mmole L⁻¹ and pH 7.4.

FIG. 13 graphically illustrates the representative distributions of the hydrodynamic diameter (based on the intensity of scattered light) of nanoparticles containing medium (13C and 13D) and high (13A and 13B) molecular weight chitosans in the presence of bovine serum albumin (BSA; 40 g L⁻¹) at time 0 (13A and 13C) and 7 (13B and 13D) hours (stability). All nanoparticles were prepared at N/P 10, with ionic strength of 150 mmole L⁻¹ and pH 7.4.

FIGS. 14A-14D graphically represent the viability of 3T3/NIH fibroblasts (14A, 14B, 14C) and RAW 264.7 macrophages (14D) treated with increasing concentrations (0.02 to 0.5 g L⁻¹) of chitosan and its DIPEA/PEG derivatives.

FIGS. 15A-15C graphically represents the viability of fibroblasts 3T3/NIH (15A, 15B) and Raw 264.7 (15C) macrophages treated with nanoparticles prepared for increasing N/P ratios and physiological conditions of pH (7.4) and ionic strength (150 mmole L⁻¹)).

FIGS. 16A-16B contain representative images of confocal macrophage microscopy RAW 264.7 treated with nanoparticles formulated by (16A) CHD32-P1.3 or (16B) CMD34 and siRNA labelled with 5′-carboxyfluorescein (FAM). CMD34 was labelled (0.6%) rhodamine isothiocyanate b (RITC) before being applied in the formulation of polyplexes. All nanoparticles were prepared at N/P 10, with ionic strength of 150 mmole L⁻¹ and pH 7.4.

FIGS. 17A-17B contains representative images of three-dimensional confocal microscopy of RAW 264.7 macrophages treated with nanoparticles formulated by CHD32 and siRNA marked with cyanine 5 (Cy5)(17A). On FIG. 17B are presented Z-stacked images of a single cell representative of the FIG. 17A.

FIGS. 18A and 18B graphically represent the transfection efficacy of anti-TNF-α siRNA transported by chitosan derivatives of medium (18A) and high (18B) molecular weight, modified with DIPEA and PEG. The study was conducted on RAW 264.7 macrophages and analyzed via ELISA-type tests. Statistical analysis(n=3) was done with unpaired Student T-tests with a statistical significance (a) of 0.05, comparing the averages of cell+LPS to the differences between the averages of cell populations. (*p<0.05, **p<0.01 and NS═Not significant).

FIGS. 19A and 19B illustrate photographs of the polymeric solutions (2 g L⁻¹) in phosphate buffer pH 7.4 with ionic strength adjusted in 150 mmole L⁻¹.

FIG. 20 illustrates the release of siRNA from CH-DIPEA₅₅ nanovectors. Analysis of siRNA preservation state by agarose gel electrophoresis after incubation of CH-DIPEA₅₅/siRNA nanoparticles with SDS at 16 mM concentration. Lines 3 and 5 show the nanoparticles formed at 10:1 and 15:1 N/P ratios. Lines 4 and 6 show the same nanoparticles after release of the siRNA in the presence of SDS.

FIG. 21 illustrates the release of mRNA from CH-DIPEA₅₅ nanovectors. Analysis of mRNA preservation state by agarose gel electrophoresis after incubation of CH-DIPEA₅₅/mRNA nanoparticles with 5 and 10% SDS. Lines 3 and 6 show the nanoparticles formed at 10:1 and 15:1 N/P ratios. Lines 4 and 7 show the same nanoparticles in the presence of 10% SDS and lines 5 and 8 with 5% SDS with the release of mRNA from the nanoparticles.

### DETAILED DESCRIPTION

### Definition

As used herein, the following designation of compounds are defined as follows:

Chitosan derivatives have adopted the following designation: chitosan (Ch) either high molecular weight (CH 100 to 300 kDa) or medium molecular weight (CM 10 to 100 kDa). When CH or CM is modified with DIPEA, such modified compounds are referred to as CHD or CMD, where the D stands for DIPEA modification of chitosan. The number added after CHD or CMD is meant to refer to the percentage of DIPEA. When such CMD or CHD is further modified with the addition of poly(ethylene glycol)(PEG), such further modified compounds are then referred to CMD-P or CHD-P. Still when a number follows the “P” designation, such number is meant to refer to PEGylation percentage (mol % relative to glucosamine units). Finally when such CMD-P or CHD-P is further complexed with siRNA, it is then referred to the nanoparticles or polyplexes.

N/P ratios as used herein is meant to refer to the ratios of moles of the amine groups of cationic polymers to those of the phosphate ones of DNA.

Ch as used herein refers to chitosan, regardless of its molecular weight.

CH as used herein refers to high molecular weight chitosan.

CHD as used herein refers to the DIPEA-CH derivative.

CHD16 as used herein refers to the DIPEA-CH derivative where the DIPEA-CH contains 16% of DIPEA.

CHD32 as used herein refers to the DIPEA-CH derivative where the DIPEA-CH contains 32% of DIPEA.

CM as used herein refers to medium molecular weight chitosan.

CMD as used herein refers to the DIPEA-CM derivative.

CMD5 as used herein refers to the DIPEA-CM derivative where the DIPEA-CM contains 5% of DIPEA.

CMD15 as used herein refers to the DIPEA-CM derivative where the DIPEA-CM contains 15% of DIPEA.

CMD34 as used herein refers to the DIPEA-CM derivative where the DIPEA-CM contains 34% of DIPEA.

CMD55 as used herein refers to the DIPEA-CM derivative where the DIPEA-CM contains 55% of DIPEA.

Ligand or targeting moieties are used herein to refer to a sequence or molecule used to target the chitosan-containing vector or the nanoparticle to a specific target. For example, without limitation but for the sole purpose of illustrating the possible targets or ligands, targeting moiety or ligand can be for example folic acid, aptamers, proteins, peptides, chimeric antibodies, humanized antibodies, or monoclonal antibodies.

The numbering of the atoms on the chitosan backbone as referred herein from time to time is the following:

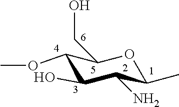

The present invention sought to synthesize biocompatible vectors comprising DIPEA-Chitosan-PEG.

This invention concerns to a process for obtaining a new vector comprising chitosan modified by varying proportions of diisopropylethylamine (DIPEA) and linked to poly (ethylene glycol) (PEG) chains) as well as the resulting derivatives and their uses. In addition, this invention concerns a process for obtaining multifunctional nanoparticles comprising these vectors as well as the nanoparticles obtained and their use for the non-viral transfer of genes, polynucleotides or derivatives thereof, for treating genetic or other diseases, which have been inherited or acquired, including through gene therapy treatments. In one embodiment, this invention concerns nanoparticles of chitosan diisopropylethylamine-PEG-siRNA-TNF-α (CH-DIPEA-PEG-siRNA-TNF-α), which can then be used for the treatment of rheumatoid arthritis, psoriasis, Crohn's disease and other inflammatory diseases. Of course this specific nanoparticle is being described here to exemplify the use of the nanoparticle, but should not be limiting the invention to this specific siRNA.

This invention also refers to nanoparticles of diisopropylethylamine-chitosan (CH-DIPEA) linked to poly chains (ethylene glycol) (PEG) and siRNA and their derived uses.

Unlike the documents mentioned above, the process of preparing the vector proposed herein comprising groups of diisopropylethylamine-chitosan-(poly)ethylene-glycol (CH-DIPEA-PEG) is simpler and allows precise control of the composition, without the quaternization of amino groups. A further advantages of the vector is that for any vector that contains PEG, such vector is more stable (up to a week at physiological pH) such that the stability now can afford the commercialization of the vector.

In a further embodiment, a ligand is added (covalently linked) to the vector to provide increased targeting, reducing side effects due to non-specificity of transfection with increased efficiency. Such ligand can be for example an aptamer or folic acid.

Still in another embodiment, there is provided a process for obtaining multifunctional nanoparticles on the basis of these derivatives and their use for gene transfer for the purpose of treating diseases of genetic or non-genetic origin, or that have been inherited or acquired, particularly by gene therapy treatment.

In a preferred embodiment, this invention involves nanoparticles of chitosan diisopropylethylamine-PEG-siRNA (CH DIPEA-PEG-siRNA), where siRNA may inhibit target cytokines such as IL-1, IL-6, RANKL, IL-17, IL-23 or TNF-α, for the treatment of arthritis, psoriasis, rheumatoid arthritis, Crohn's disease, osteoporosis, among other inflammatory or autoimmune diseases.

In a preferred embodiment, the cytokine is TNF-α, such that the nanoparticles targeting this cytokine is then chitosan diisopropylethylamine-PEG-siRNA TNF-α (CH-DIPEA-PEG-siRNA-TNF-α, for the treatment of arthritis, psoriasis, rheumatoid arthritis and Crohn's disease, among other inflammatory or autoimmune diseases.

In an alternative embodiment, this invention involves nanoparticles of chitosan diisopropylethylamine-PEG-siRNA (CH DIPEA-PEG-siRNA), where siRNA may inhibit other targets such as TyRP-1, DHT, JAK, for the treatment for example of allopecia, vitiligo and hair growth inhibition.

It is also intended to use other ligands such as aptamers or different siRNA to allow therapeutic use for the treatment of various types of cancers such as ovarian, breast or lung cancer.

It is also planned to use these nanoparticles CH-DIPEA-siRNA, CH-DIPEA-PEG-siRNA for in vitro cell transfection for the purpose of application in biomedical research or bioprocessing for recombinant proteins production.

In this invention, the nanoparticle diisopropylethylamine-chitosan (CH-DIPEA) is then linked to poly(ethylene glycol) chains (PEG) to increase stability and decrease the opsonization of the nanovector.

DIPEA and PEG grafts on chitosan results in small, positively charged nanoparticles (200 nm±100 nm) from an N/P ratio of 1<N/P<15 under physiological pH and ionic strength conditions (150 mM is the ionic strength of serum);

The addition of DIPEA to chitosan increased the degree of ionization and improve the solubility of the chitosan so modified, at physiological pH compared to unmodified chitosan or compared to the addition of DEAE to chitosan. As a result, DIPEA-chitosan is thus soluble at higher pH, such as at the physiological pH.

These results were corroborated by electrophoretic mobility studies, which indicated a strong fixation of siRNA at low load ratios (N/P 5) compared to unmodified chitosan.

Stability studies following the addition of PEG to chitosan-DIPEA indicated the presence of nanoparticles (100-200 nm) with low polydispersity (less than 0.4) 24 hours after preparation in the absence of protein and up to 7 hours after protein addition. This leads to a better stability of the nanoparticle and a lower need for siRNA for its manufacture;

In order to demonstrate the activity of the in vitro transporter system, the RAW 264.7 macrophage cell line was used. It is a widely used in vitro experimental lineage because it has reproduced characteristics very similar to rheumatoid arthritis in humans.

In the present application, the overexpression of TNF-α was induced by the use of Lipopolysacharide (LPS) and then the cells were treated with CH-DIPEA-PEG-siRNA/TNF-α as well as free siRNA-TNF-α. Confocal microscopy showed successful internalization by RAW 264.7 macrophages and analysis of TNF-α expression by macrophages revealed a suppression efficiency of up to 50% in the FBS medium. This confirms the inhibiting effect of the inflammatory response and concentration of TNF-α in the culture medium as measured by ELISA.

Vector safety has been assessed by measuring cell proliferation and STD and is between 80% and 100% cellular viability in all nanoparticle concentration conditions and in the different percentages of Ch-DIPEA-PEG tested;

Therefore, in addition to the process of obtaining diisopropylethylamine-chitosan (CH-DIPEA) derivatives linked to a binding agent (such as targeting moiety, ligands) via poly (ethylene glycol) chains (PEG) and their obtained derivatives and their uses, the purpose of one aspect of this invention is to construct multifunctional nanoparticles based on DIPEA chitosan (CH-DIPEA), containing conjugated interfering RNA (siRNA). As will be demonstrated hereinafter, the DIPEA-CH-PEG derivatives are good vectors for delivering siRNA. Hence, the person of the art, knowing what target needs to be silenced, will know which siRNA to use. The present invention is not intended to be limited to specific siRNA.

Thus, the insertion of DIPEA groups on the chitosan structure generates secondary amine groups in the polymer chain, which increases the capacity for solubility and buffering. The control of the degree of substitution and the molecular weight optimizes the effectiveness of transfection, and offers a great potential for use as a transfer agent/non-viral gene therapy.

Materials/Reagents and their Sources (Antibodies, Cell Lines . . . )

The Chitosan (Mw 200 kDa) was purchased from Polymar (Brazil).

2 kDa O-(2-mercaptoethyl)-O-methyl-polyethylene glycol (PEG-SH), 2-Chloro-N,N-diisopropylethylamine hydrochlorate (DIPEA-CI), bovine serum albumin (BSA), cellulose membrane for dialysis at WMCO 14 kDa, deuterium chloride (DCI), deuterium oxide (D20), a high-glucose culture medium D-MEM, fetal bovine serum (FBS), MISSION® siRNA universal negative control FAM labeling (siRNA-FAM), N-Succinimidyl 3-(2-pyridyldithio) propionate (SPDP), rhodamine B isothiocyanate (RITC), sodium sulphate 6 dodecyl (SDS) were purchased from Sigma-Aldrich (St. Louis, USA). EDTA sodium salt, monobase and dibase phosphate salt, potassium chloride, sodium chloride, sodium hydroxide, tris-(hydroxymethyl)-aminomethane were purchased from Dinamica (Brazil). Dimethylsulfoxide (DMSO), acetic acid, hydrochloric acid and methanol were purchased from Synth (Brazil).

The dialysis membranes of molecular weight cut-off (MWCO) 3.5 kDa and the siRNA anti-TNF-α sequence (5′-3′) sense CGUCGUAGAACCAAtt and antisense UUGGUGGUUUGCUACGACGtg were purchased from Thermo Fisher (Massachusetts, USA).

RAW 264.7 and 3T3/NIH cell lines were obtained from Banco de Celulas do Rio de Janeiro, BCRJ (Rio de Janeiro, Brazil) and ATCC™ (Virginia, USA), respectively.

The CellTiter96™ Aqueous One Solution kit consisting of 3-[4,5-dimethylthiazol-2-yl]-5-[3-carboxymethoxyphenyl]-2-[4-sulfophenyl]-2H-tetrazolium (MTS) and phenazine ethosulfate (PES) were purchased from Promega Corporation (Wisconsin, US).

The ELISA kit for TNF-α at Standard Development TMB was purchased from PEPROTECH® (New Jersey, USA).

This invention refers to the acquisition of new derivatives of diisopropylethylamine-chitosan (DIPEA-Ch) which includes the groups diisopropylethylamine (DIPEA) and O-(2-mercaptoethyl)-O′-methylpolyethylen dyle glycol (PEG-SH) in its structures.

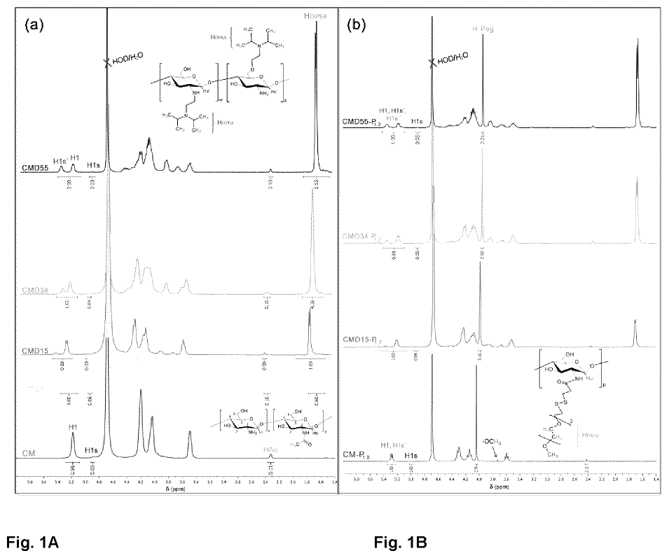

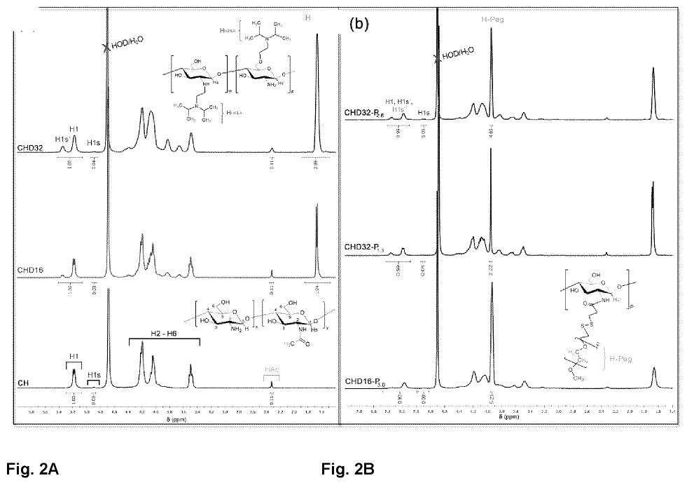

The substitution degrees for DIPEA (DS_{DIPEA}) on DIPEA-Chitosan derivatives were adjusted between 5% and 55%. It must be said that the substitution levels are determined by means of nuclear magnetic resonance with hydrogen (¹H-NMR) in accordance with FIGS. 1A-1B and 2A-2B.

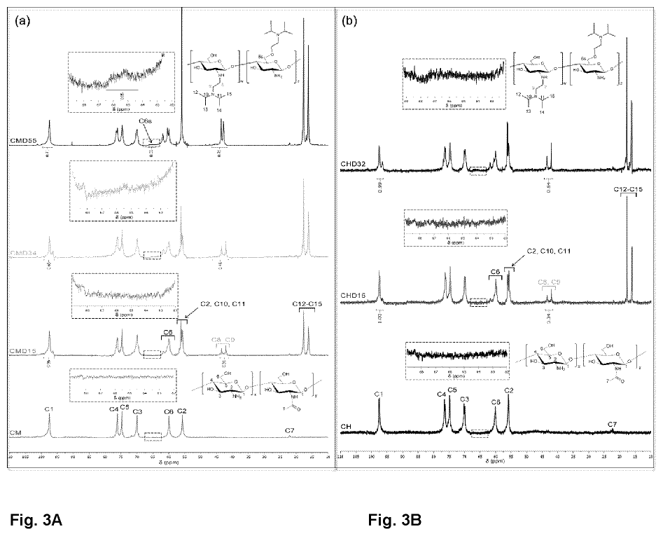

It should be emphasized that the preferred degree of substitution is between 30% and 60% of DIPEA (CHD32, CMD34 and CMD55). The DIPEA substitution degrees in carbon-related hydroxyl (6) (DSOH-DIPEA) were estimated by nuclear carbon magnetic resonance (13C-NMR), and as shown in FIGS. 3A and 3B, hydroxyl groups are minimally replaced in oxygen. The value of DSOH-DIPEA was up to 7% in the replaced DIPEA-Ch (CMD55). It is important to note that the substitution values determined by ¹H-NMR are consistent with those estimated by ¹³C-NMR (DSDIPEA-C¹³)

DIPEA-Ch derivatives with distinct DSDIPEA values were obtained by a mixture of different amounts of 2-Chloro-N,N-diisopropylethylamine hydrochlorate (DIPEA-CI) with highly deacetylated chitosan (degree of deacetylation greater than 95%), are presented in Table 1. As used hered, unless noted otherwise, chitosan of molecular weight of 100 kDa and 200 kDa were used for CM and CH, respectively. However, it has been tested and shown that other molecular weight are also acceptable and thus the present disclosure shall not be limited to these specific molecular weight.

TABLE 1
Reagents and Components for Polymer Synthesis.

| Polymer | DIPEA-Cl/Mero^{a} | PEG-SH^{b}/Mero (×10⁻²) |
|---|---|---|
| CMD5^{c} | 0.13 | |
| CMD15^{c} | 0.42 | |
| CMD34^{c} | 0.73 | |
| CMD55^{c} | 0.93 | |
| CMD15-P1.7^{d} | | 2.0 |
| CMD34-P1.3^{d} | | 2.0 |
| CMD55-P1.3^{d} | | 2.6 |
| CHD16^{e} | 0.49 | |
| CHD32^{e} | 0.93 | |
| CHD16-P3.0^{d} | | 4.0 |
| CHD32-P1.3^{d} | | 2.0 |
| CHD32-P2.6^{d} | | 4.0 |

^{a}The molar ratio between the repetition unit (mero) of the starting polymer (chitosan) and the DIPEA-Cl for the synthesis of derivatives. DIPEA-Cl: 2-cloroethyl-diisopropylamine chloride; PEG-SH O-(2-mercaptoethyl)-O′-methylpolyethylene glycol.
^{b}The molar ratio between PEG-SH and SPDP, in the synthesis of pegyled derivatives, is always 1. SPDP: N-Succinimidyl 3-(2-piridyldithio) propionate.
^{c}Medium molecular weight (CM) starting polymer (chitosan).
^{d}The pegylation of the derivatives have as their starting polymer the DIPEA-derivatives (DIPEA-Chitosan).
^{e}High molecular weight (CH) starting polymer (chitosan).

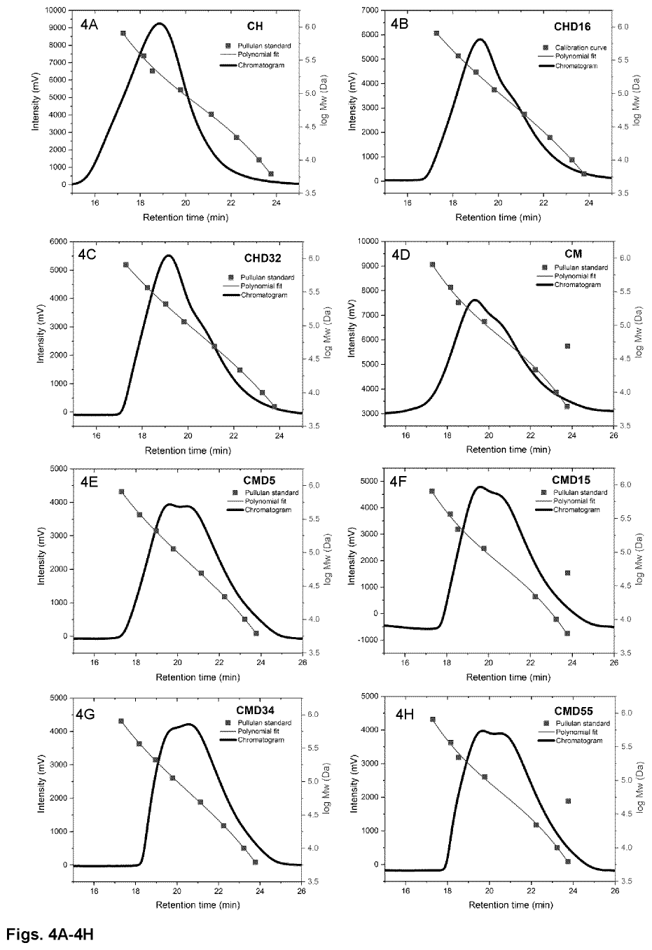

In addition, the specific substitution degrees (DSDIPEA,DSOH-DIPEA,DSDIPEA-C and DsPEG) determined by NMR, as well as the molecular weight of polymers, estimated by gel permeation chromatography (GPC), based on swarm standards (FIGS. 4A-4H) are presented in the table 2 dataset.

TABLE 2
Physicochemical properties of DIPEA-Ch.

| | DS_{DIPEA} | DS_{DIPEA-C} | DS_{OH-DIPEA} | DS_{PEG} | M̅_{w} | M̅_{w}/ |
|---|---|---|---|---|---|---|
| Polymer | (%)^{a} | (%)^{b} | (%)^{b} | (%)^{a} | (kDa)^{c} | M̅ₙ^{c} |
| CM^{d} | | | | | 141.1 | 3.02 |
| CMD5 | 5 | | | | 113.6 | 3.26 |
| CMD15 | 15 | 16 | NO^{e} | | 98.3 | 2.74 |
| CMD34 | 34 | 35 | NO^{e} | | 83.0 | 2.45 |
| CMD55 | 55 | 54 | 7.0 | | 90.6 | 2.71 |
| CMD15- | 15^{f} | | | 1.7 | | |
| P_{1,7} | | | | | | |
| CMD34- | 34^{f} | | | 1.3 | | |
| P_{1,3} | | | | | | |
| CMD55- | 55^{f} | | | 1.3 | | |
| P_{1,3} | | | | | | |
| CH^{d} | | | | | 236.7 | 1.70 |
| CHD16 | 16 | 17 | NO^{e} | | 191.6 | 2.98 |
| CHD32 | 32 | 32 | NO^{e} | | 186.0 | 2.77 |
| CHD16- | 16 | | | 3.0 | | |
| P_{2,7} | | | | | | |
| CHD32- | 32 | | | 1.3 | | |
| P_{1,3} | | | | | | |
| CHD32- | 32 | | | 2.6 | | |
| P_{2,6} | | | | | | |

^{a}Determined by ¹H-NMR. DS_{DIPEA}: Degree of substitution by DIPEA; DS_{PEG}: Degree of substitution by PEG.
^{b}Determined by ¹³C-NMR. DS_{DIPEA-C}: Degree of substitution by DIPEA via ¹³C-NMR; DS_{OH-DIPEA}: Degree of substitution at the hydroxyl groups.
^{c}Determined by gel permeation chromatography (GPC). M̅ₙ: Number average molecular weight; M̅_{w}: Weight average molecular weight; M̅_{w}/M̅ₙ: polydispersity index
^{d}Starting deacetylated chitosans (DDA 96%) of low Mw (CM) and high Mw (CH).
^{e}NO: not observed by ¹³C-NMR. The structures of the deacetylated chitosan, DIPEA-Ch derivatives with substitutions in the amine and hydroxyl groups, and the association DIPEA-Ch/PEG-SH are represented respectively by the structures 1, 2, 3 and 4 below.
^{f}Determined by ¹H-NMR for non PEGylated derivatives.

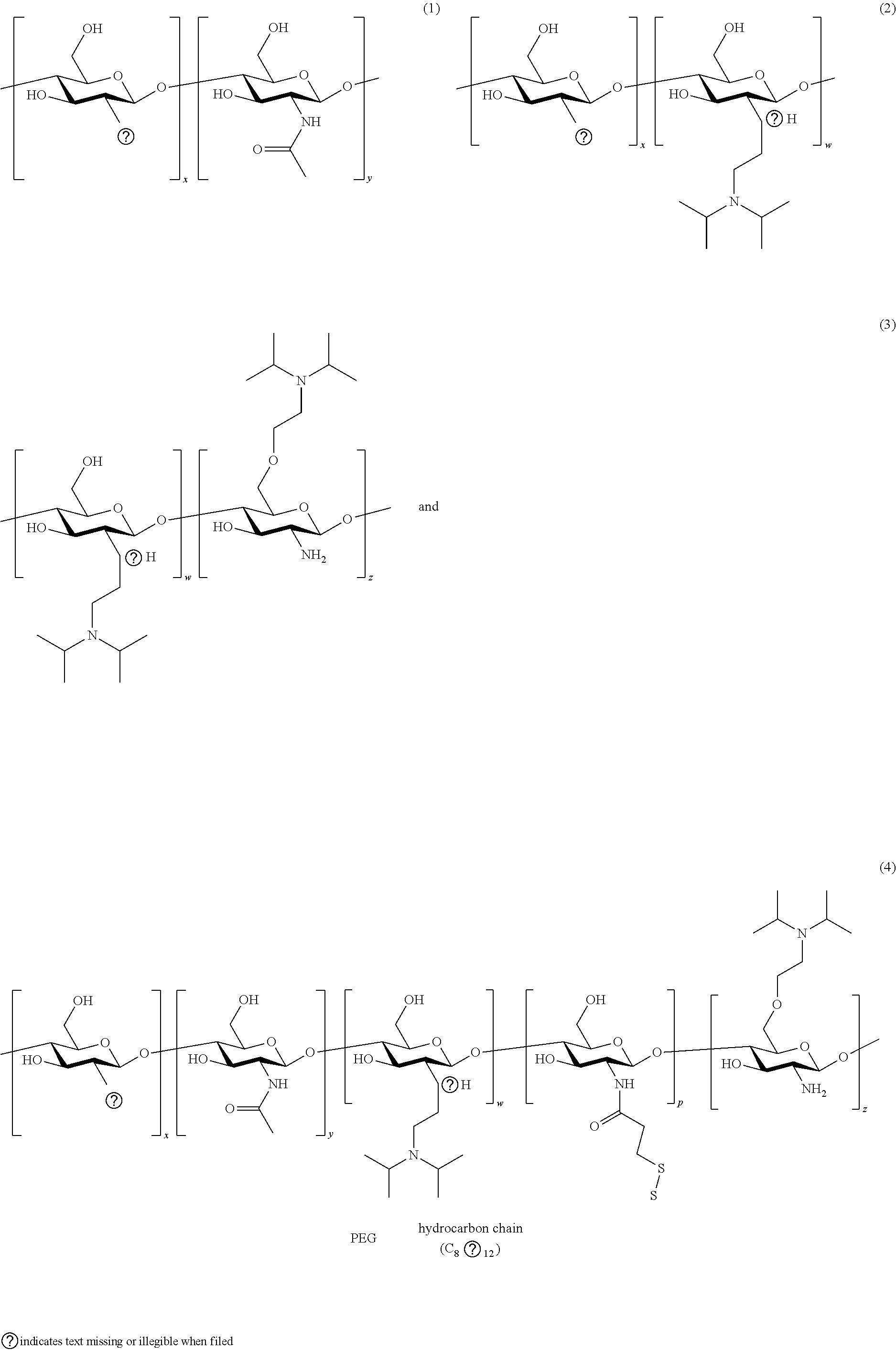

Where, the values of “p,” “w,” “x,” “y” and “z” can vary each independently from zero to one and correspond to the fraction of the repetition unit (randomly or sequentially related) in the final polymeric structure (which would contain many repeats of structure 4).

DIPEA-Ch derivatives were obtained and tested to assess their tendency to promote endosomal leakage, siRNA condensation capacity and in vitro transfection efficacy. Thus, to obtain the derivatives in accordance with an embodiment of the invention, the following reagents were used:—diisopropylethylamine-chitosan (DIPEA-Ch) with adjusted substitution degrees between 5 and 55% with molecular weight close to 100 kDa (CMD5 to CMD55) and 200 kDa (CHD16 and CHD32).

### Process of Obtaining the Derivatives

### A) Modification with DIPEA

The reactions were performed using 2-chloroethyl-diisopropylamine (DIPEA-CI) hydrochlo sustains. For the reaction, chitosan (CM or CH) was initially solubilized with a steometric amount of hydrochloric acid (HCl) 0.1 mol L⁻¹. The resulting solution was heated to 70° C. and, under constant agitation, the pH was raised to 12, adding a NaOH solution to 5 mol L⁻¹. The corresponding amount of DIPEA-CI was added to respect the molar ratio between the repetition unit (mero) of the starting polymer (chitosan) and the DIPEA-CI as reported in Table 1, and the pH adjusted again to 12. The pH of the solution was controlled throughout the reaction, adding NaOH whenever necessary to maintain the initial pH value. After 1.5 hour, the heating and agitation were stopped and the solution was cooled to room temperature. The mixture was transferred to a dialysis membrane with a 3.5 kDa molecular cut-off weight (MWCO) and dialyzed for 1 day against a solution of 0.05 mol L⁻¹ NaOH and later against deionized water, with successive changes of solvent (deionized water) to reach pH neutrality. Finally, the product (retained inside the membrane) was freeze-dried.

### B) Incorporation of PEG-SH

0.2 g of the derivative (DIPEA-CM or DIPEA-CH) was solubilized in 10 ml of acetic acid 0.03 mol L⁻¹ and the PBS 1× buffer (pH 7.4) was added, so that the final polymer concentration was 10 g L⁻¹. The pH of the solution was adjusted to 7.4 with 5 mol L⁻¹ NaOH. Sequentially, SPDP (solubilized in 1 ml of dimethylsulfoxide) was added to the DIPEA-polymer solution, which was kept agitated. After 3 hours, PEG-SH (solubilized in 1 ml of PBS 1×) was added and the reaction mixture was maintained at 40° C. for 16 hours. After this period, the product was maintained under agitation in a dialysis system (membrane with MWCO equal to 14 kDa) against PBS 1× for 3 days then against a NaOH solution with a pH between 8-9, also for three days. During dialysis, PBS was changed once a day and the NaOH was changed twice a day. Finally, the product (waterproof membrane) was recovered by freeze-drying. The amount of PEG-SH added in the DIPEA-Ch containing solution (DIPEA-polymer solution) is as shown in Table 1, the amount of SPDP compared to PEG-SH is still 1.

### Process for Obtaining the Nanoparticles

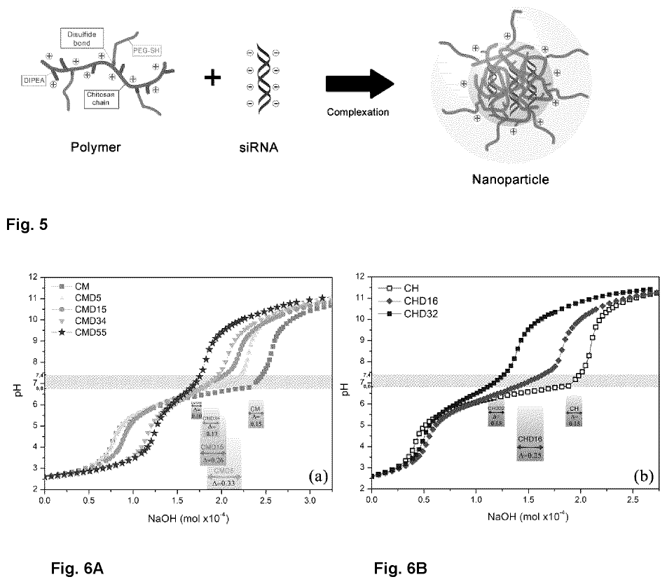

The nanoparticles were prepared using the simple complexation method, as schematically shown in FIG. 5. In this method, the self-assembly of nanoparticles is driven mainly by the electrostatic interactions that exist between the polycation (positively charged) and siRNA (negatively charged).

To this end, a mother polymer solution was initially prepared by solubilizing 2 to 4 mg of polycation in 0.2 ml of HCl 0.07 mol L⁻¹, followed by the addition of 1.8 mL of phosphate buffer pH 7,4. Then the amount de siRNA has been determined (5.0 μg for in vitro transfection and DLS studies and 0.5 μg for electrophoresis) and different volumes of the mother polymer solution were added to the nucleic acid in order to form polyplexes in different reports N/P ratio (N—amine groups; P—phosphate groups). Finally, the mixture was stirred vigorously and then left for 30 minutes in slow orbital rotation, to stabilize the newly formed polyplexes.

The volume of the nanoparticle solution depends on the characterization carried out (the N/P ration desired), and this volume is obtained by adding the same phosphate buffer used in the preparation of the polymer mother solution. N/P ratios range from 0 to 10. The 7.4 pH phosphate buffer used in the formulation of nanoparticles consists of 50 mmol L⁻¹ phosphate groups with an ionic strength adjusted to 150 mmol L⁻¹ by adding NaCl. For example, to produce CHD32/TNFα-siRNA nanoparticles for zeta potential measurement, 2.08 μL of a CHD32 solution (1.2 g L⁻¹) was mixed with 11.24 μL of siRNA (0.4437 g L⁻¹) in 1 mL of phosphate buffer to formulate polyplexes at N/P ratio 1, i.e., with the same amount (15 nmol) of amine and phophate (from siRNA) groups.

Thus, this invention also refers to nanoparticles obtained according to the process described here, in which they include in their structure DIPEA-Ch or DIPEA-Ch/PEG-SH and siRNA derivatives under N/P ratios ranging from 0 to 10.

In addition, the proposed invention refers to the uses of nanoparticles mentioned for non-viral gene therapy, as well as their combination with the controlled release of drugs of all kinds, for the treatment of genetic diseases and more particularly in the treatment of chronic inflammatory diseases mediated by TNFα and other inflammatory cytokines.

### Completed Studies

### Buffer Capacity and Degree of Ionization

Buffer capacity (CT) and degree of ionization (GI) were determined by potentiometric titration using the same procedure.

For the study, the corresponding polymer mass (previously dried) was solubilized in 40 ml of 0.01 mol L⁻¹ HCl with ionic strength adjusted to 150 mmol L⁻¹ by adding 0.35 g of sodium chloride. Then, the pH of the solution was controlled during titration with a standardized NaOH solution 0.1 mol L⁻¹. The polymer mass used in the titration was determined to obtain 2.36×10⁻⁴ moles of total amines.

The titration curves of chitosan and DIPEA-Ch derivative are shown in FIGS. 6A and 6B.

In the pH range between 7.4 and 6.8, it is observed that most derivatives had a greater or equal capacity to absorb protons compared to unmodified chitosans. This indicates a greater ability of derivatives to absorb protons during the initial pH decrease during endocytosis, promoting endosomal leakage via a mechanism called “proton sponge.”

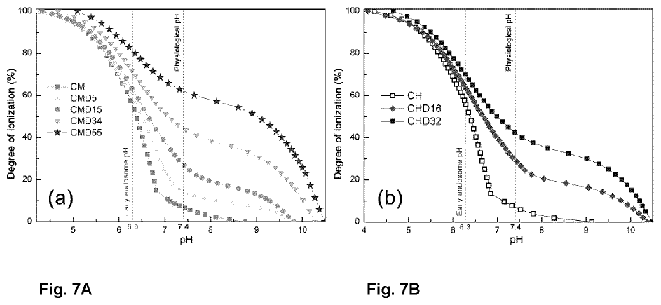

The the degree of ionization (DI) of the derivatives studied refers to the density of positive charges and is therefore directly related to the intensity of polymer interaction with nucleic acids and the surface charge of polyplexes. Chitosan DI and DIPEA-Ch derivatives are shown in FIGS. 7A and 7B.

The continuous decrease in the percentage of ionization with the increase in pH is consistent with the deprotonation of the amine groups. The insertion of substitutes (DIPEA) has led to a marked increase in the DI of derivatives at higher pH values, which have been shown to be proportional to DIPEA content. This suggests that DIPEA-Ch more effectively complex siRNA compared to unmodified chitosans at pH 7.4.

Around pH 6.3, chitosans (CM and CH) are ionized at about 50%, in accordance with the pKa value of primary amine groups in their structures.

### Release of siRNA in Agarose Gel

To prepare the polyplex (nanoparticle) solution, the amount of siRNA was set at 0.5 μg and the volume of the polyplex solution at 10 μL, of which 1.6 μL came from the load dye solution that was added to the polyplexes immediately before application on the agarose gel. The N/P ratios studied were 0.1, 2, 3, 5, 7, and 10. The agarose gel was prepared by dissolving 0.2 g of agarose in 25 ml of TAE 1×, followed by the addition of 10 μl ethidium bromide (10 g L⁻¹) The running was done under a potential of 80 volts for 1 hour and 15 minutes, using the TAE 1× as an electric conductor.

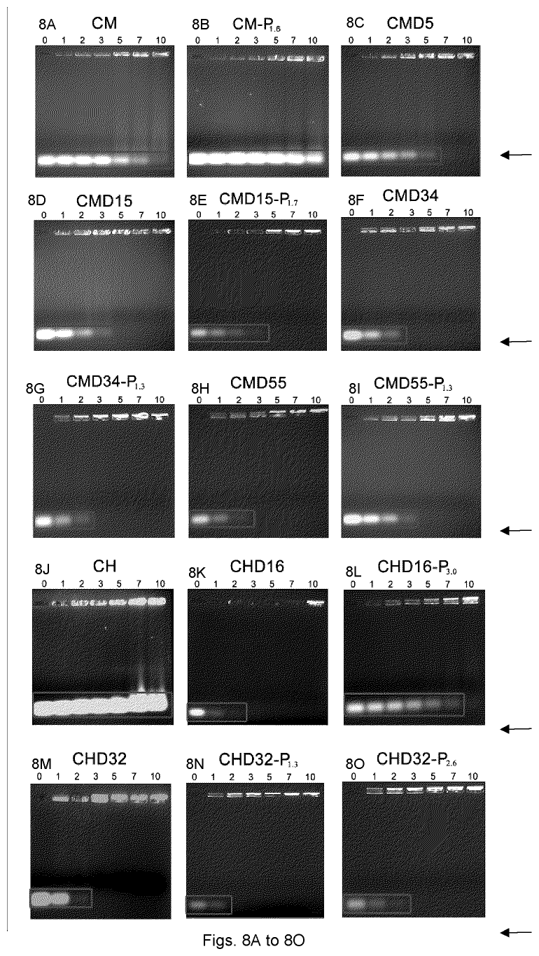

The study of the release of siRNA in agarose gel allows qualitative evaluation of the condensation efficiency of siRNA by polycations (DIPEA-Ch-PEG). Photographs of agarose gels under ultraviolet light are shown in FIGS. 8A-80.

Polyplexes prepared with unmodified chitosans released siRNA in all N/P ratios studied, indicating low capacity of these polymers to act as vectors under the physiological conditions of the study.

However, the modification with DIPEA clearly increased the condensation efficiency of derivatives (compared to unmodified chitosan) with complete siRNA retention in wells from the N/P 3 ratio.

In addition, PEGylation reduced the condensation efficiency of some DIPEA-Ch derivatives, increasing the minimum N/P ratio in which there is complete siRNA retention in agarose gel wells.

Comparing derivatives of the same composition, but of different molecular weight (CMD15 and CMD34 versus CHD16 and CHD32), clearly shows that the increase in molecular weight (MW) promotes an increase in the strength of interaction with siRNA.

### Zeta Potential(ζ), Hydrodynamic Diameter (Dh) and Polydispersity of Nanoparticles

The amount of siRNA used herein was set at 5 μg and the volume of the DIPEA-Ch polyplex solution prepared at PBS pH 7.4 and ionic strength 150 mM was 1 ml. The hydrodynamic diameter and polydispersity of the polyplexes (DIPEA-Ch-PEG-siRNA TNFα) were analyzed in the N/P 1, 3, 5, 7 and 10 ratios. Each N/P ratio was tripled and each was measured 3 times in a Zetasizer NanoZS (Malvern Instrument). The results of the hydrodynamic diameter (Dh) were expressed on the basis of the average Z. The nanoparticles were prepared in triplicata and each was analyzed 3 times, the results being expressed as (average±standard deviation).

Nanoparticles, in addition to an adequate size (usually less than 500 nm), need a minimum positive zeta potential (ζ) (at pH 7.4) to promote cell absorption and, therefore, transfection efficiency.

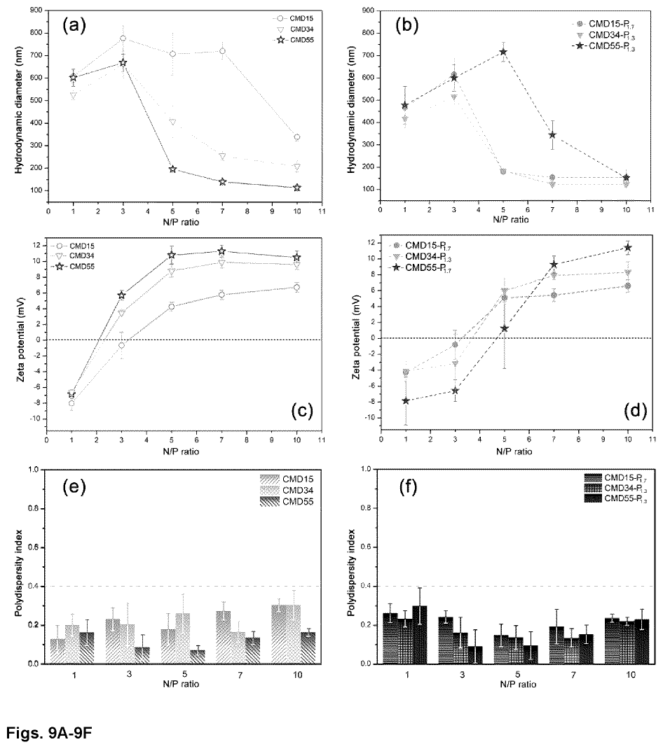

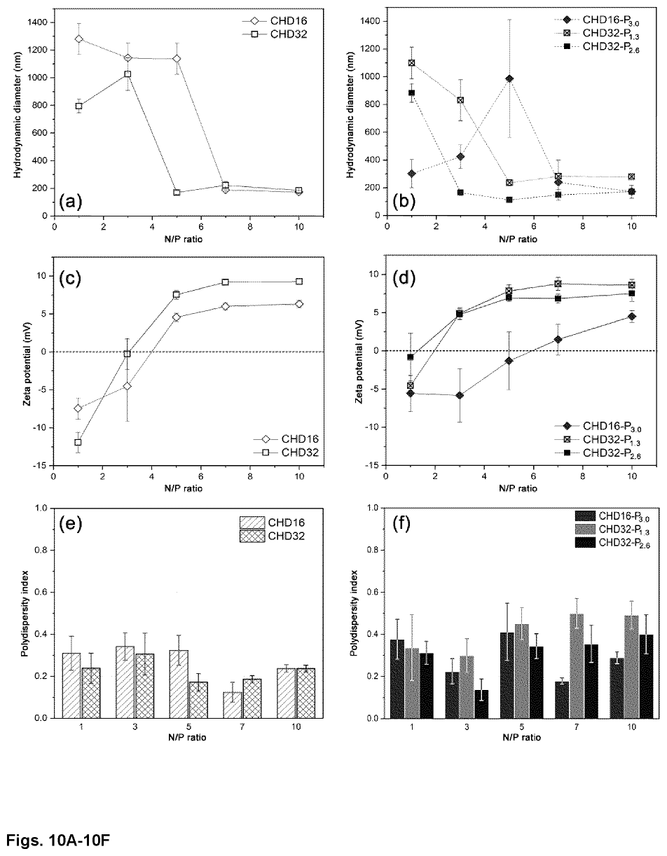

The hydrodynamic diameter (Dh), the potential (and the polydispersity (PDI) of polyplexes formed by medium and high MW polymers, based on the N/P ratio, are represented in FIGS. 9A-9F and 10A-10F.

In general, it is observed that the higher the DSDIPEA, the lower the N/P ratio is required for the formation of nanoparticles and the greater the potential (of polyplexes. Such proportionality (between DSDIPEA and the surface load of nanoparticles) corroborates with the ionization degree of derivatives at pH 7.4.

It should also be noted that nanoparticle formation (less than 400 nm) occurs only in N/P ratios where the potential ζ is positive, indicating that the packaging is only effective when the (negative) siRNA load is completely neutralized.

Comparing medium to high molecular weight (MW) derivatives shows that the increase in molecular weight allows nanoparticles to be obtained in lower N/P ratios. For example, while CMD34 formed nanoparticles with Dh of about 200 nm only in the N/P ratio 10, CHD32 formed nanoparticles with the same Dh in N/P 5 ratios. This indicates the presence of other interactions, in addition to electrostatic, in the formation of polyplexes.

In addition, for most derivatives, the PEG graft reduced the amount of polymer needed to formulate nanoparticles with 150-200 nm Dh by up to 50%. In most N/P ratios, PEGylation also caused a decrease in the module of potential values (polyplexes.

Therefore, this demonstrates the importance of adding DIPEA (PEG-associated) to chitosan, as nanoparticles with Dh around 200 nm and positive zeta potential were obtained under physiological conditions of pH and ionic strength, under lower N/P ratios.

### Morphological Evaluation by Scanning Electron Microscopy with Field Emission Cannon (SEM-FEG)

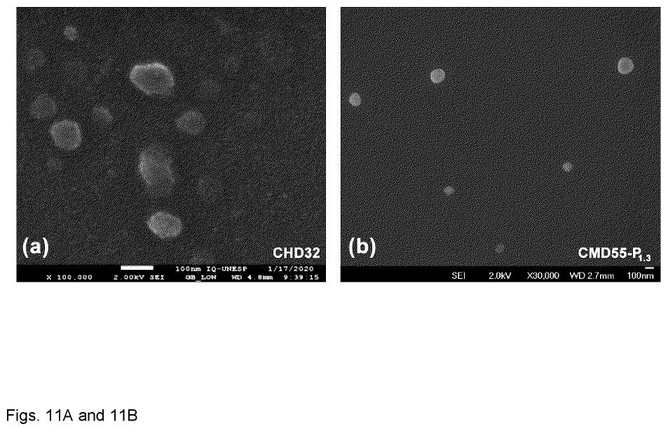

The nanoparticles were prepared in the N/P 10 ratio, as described above. Sequentially, 1 μL of the nanoparticle solution was applied to the surface of a silicon plate, which was kept at room temperature inside a dryer for solvent evaporation. The vectors were then analyzed by scanning electron microscopy (SEM) with an electron emission cannon of the FEG type (Field Emission Gun) in a JSM-6701F (JEOL) microscope. Samples were examined under an acceleration voltage of 2.0 kV. SEM-FEG representative images of nanoparticles formed by CHD32 and CMD55-P1.3 under physiological pH and ionic strength conditions are presented in FIGS. 11A and 11B.

DIPEA-Ch derivatives formed DIPEA-Ch-siRNA nanoparticles of sphere-like morphologies.

In addition, the SEM-FEG results indicate slightly smaller dimensions than those reported by the diffusion of light (i.e. hydrodynamic diameter), in accordance with the dry state of the nanoparticles during this analysis.

### Colloidal Stability of Nanoparticles Under Physiological Conditions of pH and Ionic Strength

The nanoparticles obtained in the N/P 10 ratio had their Dh and polydispersity (PDI) tracked over time. The amount of siRNA and the final volume of the polyplex solution are the same as those described above. The times studied were: 0; 0,5; 2; 4; 7; and 24 hours, and between analyses, the nanoparticles were kept under low orbital agitation at 37° C. The nanoparticles were duplicated and analyzed 3 times each. The results were expressed in (average±standard deviation).

In the N/P 10 ratio, all derivatives have nanoparticles with good Dh results, a potential zeta and a siRNA protection capability, well, this was the N/P ratio chosen for stability studies.

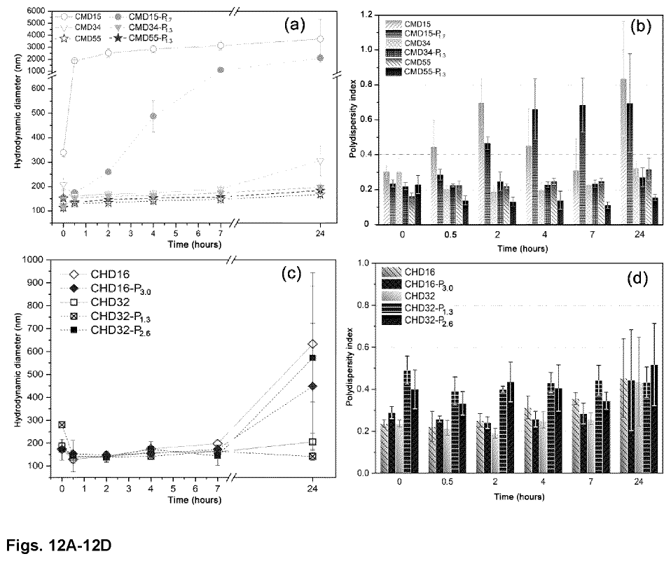

The variation of Dh over time depended on DSDIPEA, the presence of PEG and molecular weight (Mw), as shown in FIGS. 12A-12D.

The increase in DSDIPEA and MM promoted greater stability in the polyplexes, focusing on the nanoparticles obtained by CMD55 and CHD32 which, after 24 hours, still showed Dh around 200 nm.

In the derivatives of the average MM, the addition of PEG increased the colloidal stability of the polyplexes, with the emphasis on derivative with DS-DIPEA by 15% (CMD15 versus CMD15-P1.7).

In general, the polydispersity index (PDI) of all polyplexes has increased over time and, given CMD15 and CMD55, this increase is inversely proportional to the DSDIPEA. Polyplexes formed by medium molecular weight (CM), with the exception of CMD15 and CMD15-P1.3, kept the polydispersity index (PDI) below 0.4 during up to 24 hours of study.

### Colloidal Stability of Nanoparticles in the Presence of Albumin (BSA)

In this study, the amount of siRNA was set at 5 μg and the volume of the nanoparticle/BSA solution at 1.1 mL. The distribution of nanoparticle size was assessed immediately (zero hour) and seven hours after the application of BSA (40 g L⁻¹), and between analyses, the nanoparticles were kept in low orbital agitation at 37° C. The nanoparticles were duplicated and each was analyzed 3 times, but only one of the two sets of analyses was represented in the form of a distribution curve to promote comparison between the results obtained.

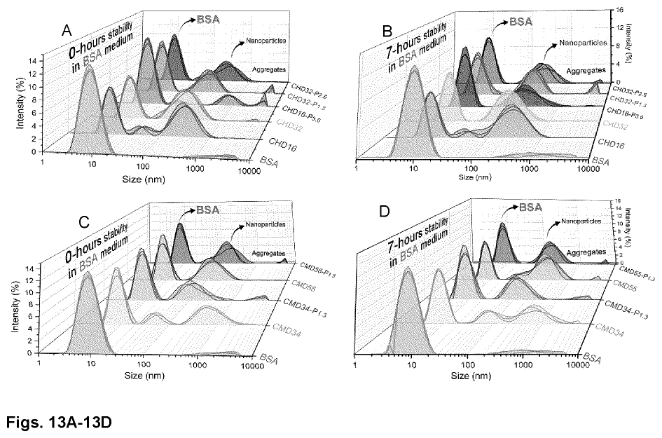

The Dh distribution curves based on the intensity of light scattering (over time) by the sample constituents are shown in FIGS. 13A-13D.

The BSA has a Dh of about 8 nm. When nanoparticles interact with BSA, there is a clear change in the distribution of Dh (i.e. signs referring to larger structures), suggesting the formation of aggregates.

The DIPEA graft, molecular weight and PEG transplants have influenced the formation of polyplex/BSA aggregates.

For nanoparticles formed by CMD55, the presence of aggregates was not observed. The polyplexes obtained with CMD34, on the other hand, formed aggregates with BSA immediately after mixing. However, when the nanoparticles were prepared with the PEGylated version of this derivative (CMD34-P1.3), an inhibition of aggregate formation was observed up to 7 hours in the study.

In MW derivatives (CMD), no aggregate formation was observed within 7 hours, regardless of the presence of PEG. This suggests the presence of other interactions (in addition to electrostatic) in vector stabilization in a simulated biological environment.

### Evaluation of the Cytotoxicity of Polymers and Nanoparticles

Cell viability in the presence of polymers was determined with the cell proliferation commercial kit CellTiter96™ AQueous-One Solution (Promega Corporation), consisting of 3-(4,5-dimethylthiazol-2-yl)-5-(3-carboxymethoxyphenyl)-2-(4-sulfophenyl)-2H-tetrazolium (MTS) and phenazine ethosulfate (PES).

The cells used in the tests were fibroblasts of the 3T3/NIH lineage and macrophages of the RAW 264.7 lineage. To this end, the cells were initially grown in 96-well plates at a density of 2×10⁴ cells/wells (in 200 μL complete medium) and kept in humidified incubation (37° C. with an atmosphere of 5% CO₂) for 24 hours. After this period, the medium was removed and 200 μL of polymeric solutions (in the complete medium) were added to concentrations of 0.02; 0,05; 0,1; 0.2; and 0.5 g/L⁻¹. As positive and negative controls of cytotoxicity, 3% (p/v) sodium dodecyl sulfate (SDS) and untreated cells, respectively, were used. Again, the cells were kept in incubation for 24 hours, then the medium on these was removed and 100 mL incomplete medium (DMEM only) were added, followed by the addition of 20 MTS/PES. After an incubation period of 3 hours, well absorption was measured in a plate reader (Biotek Elx 808™) at a wavelength of 490 nm. The percentage of cellular viability was determined as shown below.

$$Cell Viability ( \% ) = \frac{( {A}_{sample} - {A}_{negative control} )}{( {A}_{positive control} - {A}_{negative control} )} \times 100$$

Where Aₛₐₘₚₗₑ, A_{negative control} and A_{positive control} refers to absorbances of treated cells, untreated cells and cells treated with 3% of sodium dodecyl sulfate, respectively. The studies were conducted in three-way and the results were expressed in (average±standard deviation).

To prepare the polymeric solutions applied to the cells, a polymer stock with a concentration of 2.5 g L⁻¹ was initially prepared. For this purpose, 5.0 mg of the polymers (DIPEA-Ch or DIPEA-Ch-PEG) were solubilized in 0.2 mL of 0.1 mol L⁻¹ of HCl followed by the addition of 1.8 mL of complete medium. For example, an acidic control (containing only HCl and the complete medium) was also used to determine possible effects on cells.

Cell viability in the presence of nanoparticles has been achieved in a manner similar to viability in the presence of polymers. To prepare the polyplexes, the amount of siRNA was set at 0.55 μg and the volume of the polyplex solution at 50 μL. Once stabilized (i.e. after 30 minutes of orbital agitation), the nanoparticle solution (50 μL) and 150 μL of complete medium were applied to the cells. The N/P ratios studied were: 1, 5, 10, and 20. Controls of free siRNA and phosphate buffer at pH 7.4, prepared and used under the same conditions as polyplexes, were also used.

Two cell lines were used to assess the cytotoxicity of the polymers and nanoparticles: 3T3/NIH fibroblasts and RAW 264.7 macrophages.

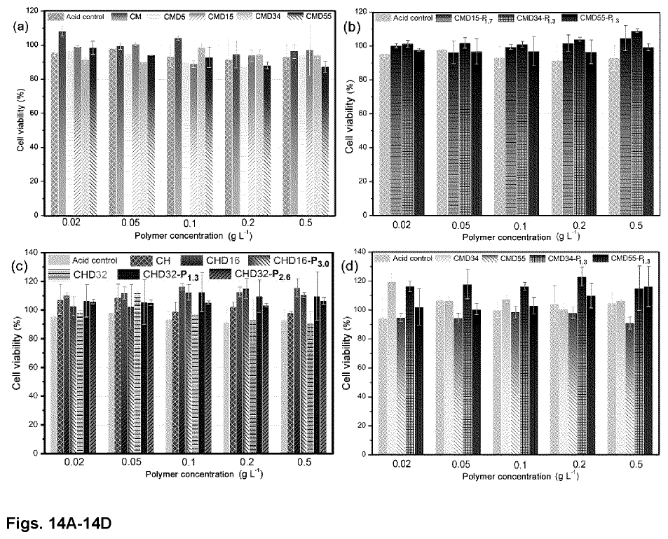

Cell viability was assessed in the presence of increasing concentrations of the polymers, as shown in FIGS. 14A-14D.

It is observed that for all polymers studied, cell viability was greater than 80%, regardless of concentration and cell line. The control of the acid was found to have no effect on the dosage. These results indicate that even with chemical changes, derivatives maintained the non-cytotoxic characteristics of chitosan. In addition, PEGylation did not promote changes in the cytotoxicity of polymers.

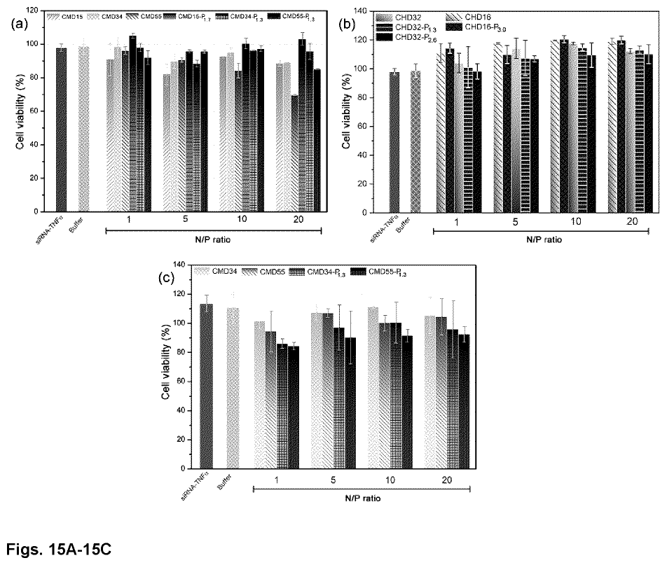

Cell viability studies in the presence of nanoparticles are presented in FIGS. 15A-15C.

In fibroblasts, up to the N/P 10 ratio, all polyplexes derived from medium molecular weight polymers showed viability of 80% or greater. However, in the N/P 20 ratio, it is possible to observe a decrease in cellular viability (to 70%) when nanoparticles derived from CMD55 were applied to the cells. For this same polymer (N/P ratio and cell line), it can be observed that the addition of PEG (CMD55-P1.3) promotes a decrease in the cytotoxicity of polyplexes (cell viability is greater than 80%).

In macrophages, all polyplexes studied showed cellular viability of 80% or greater, regardless of the N/P ratio. The cellular viability of fibroblasts (3T3/NIH) in the presence of nanoparticles derived from high molecular weight polymers (FIG. 15C) was greater than 85%, regardless of the N/P ratio, DSDIPEA and DSPEG.

It is reported that nanoparticles consisting solely of chitosan have high cellular viability (above 80%), even at higher concentrations. Again, in this study, it was indicated that nanoparticles maintain the biocompatibility characteristics of chitosan.

### Cell Internalization Study by Confocal Microscopy Analysis

In this study, the internalization of nanoparticles formed by siRNA marked with cyanine5 (Cy5) or 6-carboxyfluscein amidite (FAM) was evaluated. The polymers used in the study to obtain two-dimensional (2D) images were CHD32-P1.3 and CMD34, the latter being marked with 0.5% (mol/mol) rhodamine B isothiocyanate (RITC). The polyplexes formulated by CHD32 were analyzed by three-dimensional confocal microscopy (3D).

The nanoparticles (1 ml) were prepared in the N/P 10 ratio and the amount of siRNA was set at 5 μg.

The cells used in the test were macrophages of the RAW 264.7 line, plated on glass slats previously placed in the wells of a 6-well plate. The cells were seeded at a density of 3.8×10⁵ cells/wells (in 4 ml of complete medium) and kept in humidified incubation (37° C. with an atmosphere of CO₂ to 5%) for about 20 hours. After this period, the medium was removed and 1 ml of the nanoparticle solution and 3 ml of incomplete medium (without antibiotics and FBS) were added to the cells, which were kept in incubation for 4 hours. After this period, the medium was removed and the cells washed with PBS to be fixed with 4% (1 ml) of p-formaldehyde (PFA) for 15 minutes and colored with (1 ml) 4′,6-diamidino-2-phenylindole (DAPI) 1 mg L⁻¹ for 10 minutes. Fixing with PFA and coloring the nucleus with DAPI were performed at room temperature. The cells were washed with 1×PBS after the addition of PFA and DAPI. The slats were then placed on glass blades and the images captured using a Zeiss confocal microscope model LSM 710™ (ZEN software 2010).

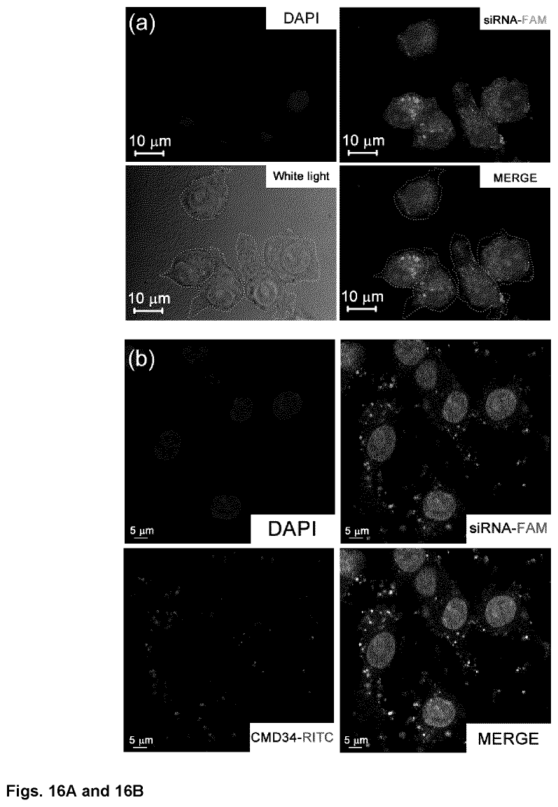

2D microscopy images (FIGS. 16A-16B) indicate a high rate of cell capture of polyplexes, as green fluorescent spots (siRNA-FAM) are observed throughout the cell area four hours after nanoparticles are applied to cells.

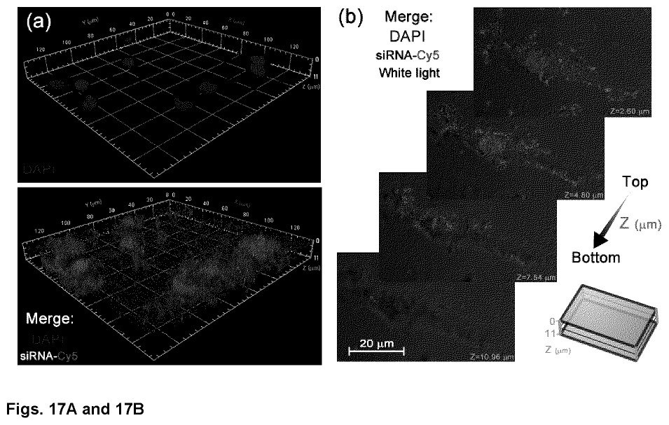

In addition, the results indicate a continued release of siRNA, as there are isolated green dots (free siRNA) and red fluorescent dots (marked polymer) co-located at the same time (FIG. 16B). In order to rule out the possibility that the adhesion of nanoparticles to the cell surface causes an overestimation of vector internalization, a 3D microscopy of macrophages treated with polyplexes formulated by CHD32 was also performed, as shown in FIGS. 17A-17B.

In 3D microscopy, high levels of siRNA (red dots) are observed in all Z-axis values, highlighted in Z-stacked images, confirming the effective internalization of nanovectors by macrophages.

### In Vitro Transfection Study

Transfection efficacy was determined by quantifying the TNFα protein produced by RAW 264.7 macrophages, using the Murine TNF-α Standard TMB ELISA Development (PEPROTECH™) commercial kit.

The cells were initially grown in 24-well plates at a density of 1.8×10⁵ cells/wells (in 1 ml of complete medium-DMEM supplemented with 10% FBS and 1% antibiotic/antimycotic solution) and maintained in humidified incubation (37° C. with an atmosphere of 5% CO₂) for about 20 hours. After this period, the medium was removed and the cells were washed (twice) with 1 ml of incomplete medium. Then, 0.45 ml of the nanoparticle solution (equivalent to 5 μg of anti-TNFα siRNA) and 1.35 ml of medium (with or without FBS, but still without antibiotics) were added to the cells. Again, the cells were kept in incubation for 5 hours, then the medium on them was changed to 1 ml of complete medium. 24 hours after the start of transfection (i.e. the application of nanoparticles on cells), the medium was exchanged for 0.3 ml of complete medium containing Escherichia coli lipopolysaccharide (LPS) at a concentration of 100 mL⁻¹, to stimulate the production of TNFα. The cells were kept in incubation for a period of 4 hours and subsequently, the supernatant was removed, centrifuged (12 000 g) and stored (−20° C.) for subsequent quantification of TNFα via an immuno-enzymatic dosage (ELISA), as instructed by the manufacturer.

In this study, the amount of anti-TNF-α siRNA was set at 5 μg and the volume of the polyplex solution was 0.45 ml. The N/P ratios studied were: 1, 5 and 10. Controls of free siRNA, cells treated only with LPS (Cell-LPS) and cells without any type of treatment (Cell) were also evaluated.

The ELISA test consists of several steps, the final procedure of which is a color assessment which, in this case, indicates the amount of TNFα present in the sample. For analysis, the samples (cell supernatant) were previously diluted (1:40) in PBS and the amount of TNFα was expressed in relation to the absorption (at 450 nm) of the cells treated with LPS (A-(Cell-LPS)), as shown in equation 13.

$$Expression TNF α ( \% ) = \frac{( {A}_{sample} )}{( Acells LPS )} \times 100 \qquad 13$$

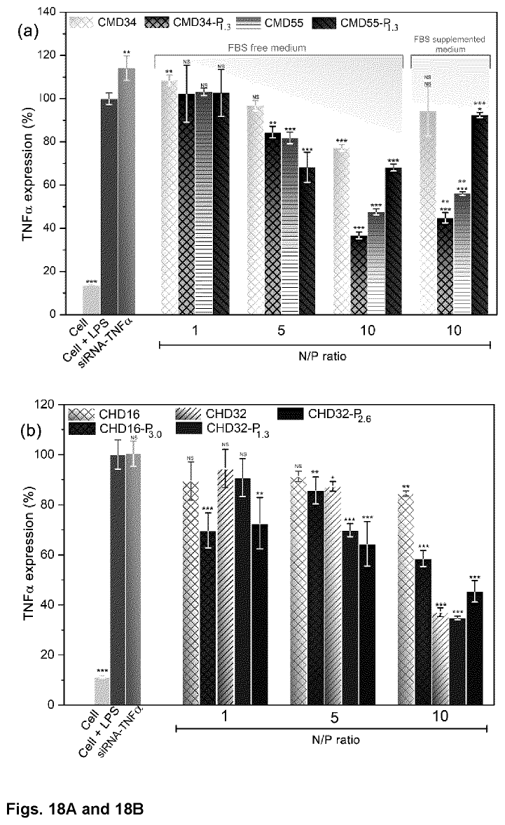

It should be noted that in the transfection study, the same concentrations of the cytotoxicity test of nanoparticles were determined so that conditions with a high rate of cellular viability could be applied (in the transfection study). The studies were conducted in three-way and the results were expressed in (average±standard deviation), as is graphically shown in FIGS. 18A-18B. The higher the relative expression of TNFα, the lower the transfection efficacy of the studied polyplex.

The results were found to be dependent on the N/P ratio, on the DSDIPEA, of the molecular weight and of the presence/absence of PEG.

When the N/P ratio increases, there is a decrease in the expression of TNFα. This was expected, because there are the appearance of characteristics favorable to gene transfection with an increase in the ratio N/P, such as: formation of a positive surface load and decrease of Dh in nanoparticles, in addition to increasing the ability of the polycation to offer protection to siRNA. Thus, the N/P 10 ratio showed the highest transfection rates and, therefore, this ratio was established to assess the effect of other variables (DSDIPEA, MW and PEGylation).

For polymers of the same molecular weight (MW), there is a proportionality between DSDIPEA and transfection efficiency. For example, in high Mw polymers (CHD16 vs. CHD32), twice the amount of DIPEA in chitosan halved the level of expression of TNFα. A similar effect was observed in polymers of the same composition, but with different MW (CMD34 versus CHD32). By doubling the molecular weight (MW) of the derivative, the transfection efficiency of nanoparticles has been doubled.

Again, these results are consistent with the characteristics of the nanoparticles obtained to date. The increase in DSDIPEA and/or MW has led to the formation of more compact and stable nanoparticles with positive zeta potentials, desirable attributes for cationic polymer-based nanovectors.

The addition of PEG increased (CMD34 and CHD16) or did not alter (CHD32) the transfection efficiency of polyplexes. It should be noted that CMD34 pegylation has doubled its effectiveness as a transfection agent.

It can also be observed that, even in the presence of FBS, the CMD34-P1.3 and CMD55 derivatives reduced TNFα expression by 50-60%, compared to LPS-stimulated cells. This confirms the stability results in the presence of BSA presented above.

### Protection Capacity of the Chitosan-DIPEA Nanovector

CH-DIPEA₅₅/siRNA-anti TNF-α nanoparticles at N/P ratio of 10:1 and 15:1 were prepared in PBS and stored at room temperature for 8 months in the dark. After this period, the nanovector protection was evaluated on a 4% agarose gel. For this, nanoparticles were incubated with sodium dodecyl sulphate (SDS, 16 mM) to allow the release of the cargo, thus, the qualitative analysis of siRNA integrity was carried out compared to a fresh free siRNA used as a control.

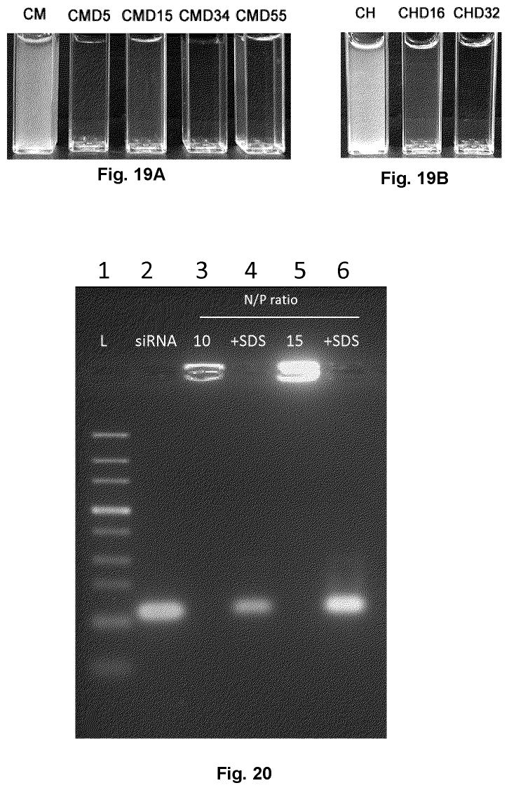

FIG. 20 shows the state of conservation of siRNA-anti TNF-α released from the nanovector after 8 months of complexation. The columns 3 and 5 show the nanoparticles inside the well confirming the stability of the systems. In presence of SDS, these nanoparticles released their nucleic acid (columns 4 and 6). It is possible to observe that the migration pattern in the agarose gel and the band intensity of the released siRNA (formerly complexed in the nanovector) and the free siRNA (control, second column) were nearly the same, confirming the good conservation state of the payload.

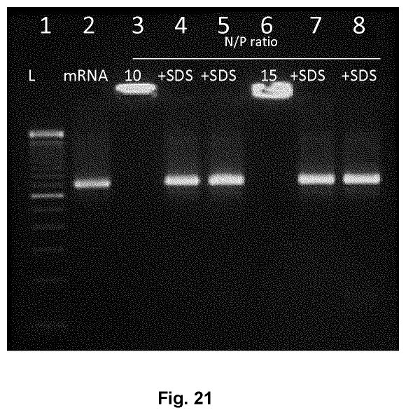

A similar protocol was being carried out with CH-DIPEA₅₅/mRNA-eGFP nanoparticles. So far, the complexation of mRNA-eGFP by CH-DIPEA₅₅ nanovector has been validated on agarose gel. As well, the use of SDS to release the payload from the NV has been observed. FIG. 21 shows CH-DIPEA₅₅/mRNA-eGFP nanoparticles prepared at N/P ratios of 10:1 and 15:1 in the presence of different concentration of SDS (5% and 10%). To validate the conservation state of mRNA over a longer period of time, the nanoparticles freshly prepared will be evaluated at a later time.

The potential application of CH-DIPEA₅₅ as a nanovector to complex and protect nucleic acids over time at room temperature becomes apparent from FIG. 20. This characteristic confers an inestimable value, as this nanoplatform may be used not only as a vector for transfection but also for some applications such as vehicle and delivery system for vaccines offering protection from degradation. This fact offers the possibility to protect payloads without the burden of a cold chain process in transport, storage and handling, as it is necessary for some immunizing agents nowadays.

### Advantages of the Present Invention

There are major advantages of DIPEA Versus DEAE derivatives when combined to chitosan nanoparticles that grant a unique profile to these new vectors.

Although prior derivatives of chitosan (reported to be useful for transfection) were toxic to cells. In order to address the cytotoxicity problem, derivatives of chitosan with diisopropylethylamine (DIPEA) groups linked to the main chain have been synthesized.

Further, the presence of DIPEA increases the stability and solubility of nanoparticles in physiological pH. The presence of PEG increases solubility, decrease opsonization and increase the bioavailability of the nanoparticle. As mentioned above, The addition of DIPEA to chitosan increased the degree of ionization and improve the solubility of the chitosan so modified, at physiological pH compared to unmodified chitosan or compared to the addition of DEAE to chitosan. As a result, as can be seen on FIGS. 19A and 19B, DIPEA-chitosan is thus soluble at higher pH, such as at the physiological pH, more than chitosan alone. As can be appreciated from the photographs of FIGS. 19A and 19B, CM and CH at pH 7.4 is troubled and cloudy showing the insoluble character of chitosan in suspension. The more DIPEA is added the clearer the solution becomes, showing the improved solubility at pH 7.4.

The production of the DIPEA-Ch vectors becomes much simpler compared to other chitosan-containing vectors. Contrary to DEAE-CH derivatives, the synthesis of DIPEA vectors does not lead to quaternized units, since the voluminous isopropyl amino groups cannot be substituted. Hence, undesirable side reactions are avoided, and the compositions can be easily tuned in. This is a major strategic advantage in future industrial production with a significant cost-saving impact.

Further, contrary to other chitosan-containing vectors such as DEAE-CH, DIPEA modifications to the chitosan backbone provide for a much lower need for chitosan deacetylation (down to 88% from 98%) in order to preserve the same low cytotoxicity and high transfection efficacy. This chemical characteristic sets the DIPEA-Ch conjugate uniquely apart from all previous chitosan nanoparticles that need such deacetylation.

In summary, compared to chitosan and DEAE-Ch, DIPEA conjugation to chitosan has generated a remarkable effect on the chemical structure (less deacetylation, less quaternization), a much better functionalization of its synthesis (better “tune-in”, no side-reactions, easier and more cost-effective production), significantly improved physico-chemical characteristics (nanoparticle stability of up to 1 week at pH 7.4, less aggregation when exposed to albumin, higher solubility at physiological pH, high solubility when exposed to albumin and serum), and full biodegradability of its components. It has also demonstrated outstanding low-to-no cytotoxicity in vitro and enhanced transfection and siRNA activity under serum exposure.

These results significantly differentiate these new DIPEA-Ch from previous non-modified or DEAE-CH modified nanoparticles both in its structure, its functionalization, its synthesis, its physico-chemical properties, its biological activity in vitro and its potential in vivo application.

As can be seen therefore, the examples described above and illustrated are intended to be exemplary only. The scope is indicated by the appended claims.

1. A chitosan-containing vector as set forth in formula I: 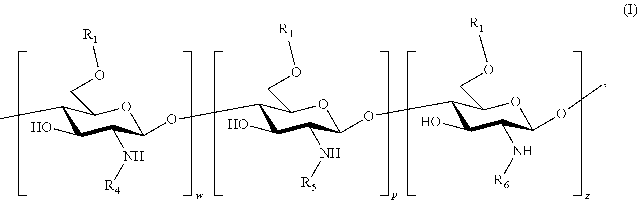
wherein
R₁, R₂, and R₃ are each independently hydrogen or 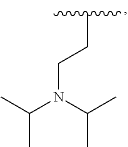 [SMILES: *CCN(C(C)C)C(C)C]
where [symbol] represents a point of attachment,
R₄, R₅ and R₆ are each independently hydrogen, —C(O)—CH₃, —C(O)CH₂—(O—CH₂—CH₂)ₙ—R₇, —C(O)CH₂—CH₂—R₈, or 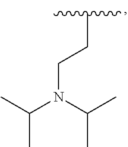 [SMILES: *CCN(C(C)C)C(C)C]
R₇ is hydrogen, hydroxyl, a ligand or a targeting moiety,
R₈=is —S—S—R₉
R₉ is a (C₈-C₁₂)Alkyl or —CH₂—CH₂—(O—CH₂—CH₂)ₙ—OR₁₀,
R10 is hydrogen, a ligand or a targeting moiety,
n is an integer between 0 and 50,
w, p, and z are integers varying from 0 to 1500, the sum of w, p, and z being between 50 and 1500, so as to define a chitosan backbone with a molecular weight between 10 kDa and 300 kDa,
said chitosan-containing vector comprising 5% to 55% of 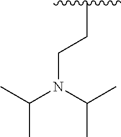 [SMILES: *CCN(C(C)C)C(C)C]
relatively to the sum of w, p, and z, and up 5% for pegylated units.

2. A chitosan-containing vector comprising a backbone comprising diisopropylethylamine (DIPEA) covalently linked to the 6-hydroxyl group of chitosan or to the amino group on the C2 position of Chitosan.

3. The chitosan-containing vector of claim 2, wherein said chitosan has a molecular weight comprised between 10 kDa and 300 kDa, with a degree of deacetylation varying from 70% to 99%.

4. The chitosan-containing vector of claim 2, wherein the hydrogen of the hydroxy group on C6 or of the amine group on C2 is substituted between 5 and 55% with DIPEA.

5. The chitosan-containing vector of claim 2, wherein the backbone further comprises polyethylene glycol (PEG) covalently attached directly or through a disulfide bridge to the amino group attached to position C2 of the chitosan.

6. The chitosan-containing vector of claim 5, wherein the PEG has a molecular weight varying between 2 kDa and 10 kDa.

7. The chitosan-containing vector of claim 5, wherein the hydrogen of the amine group on C2 is substituted between 2 and 5% with PEG.

8. The chitosan-containing vector of claim 2, wherein said backbone further comprises a ligand or a targeting moiety.

9. The chitosan-containing vector of claim 8, wherein the ligand or targeting moiety is selected from the group consisting of folic acid, aptamers, proteins, peptides, chimeric antibodies, humanized antibodies, and monoclonal antibodies.

10. A nanoparticle comprising the chitosan-containing vector of claim 1 and a polynucleotide.

11. The nanoparticle of claim 9, wherein the polynucleotide is selected from the group consisting of interference RNA (siRNA), messenger RNA (mRNA) or DNA.

12. A method for preparing a chitosan-containing vector, said method comprising the steps of:
reacting chitosan (Ch) with 2-Chloro-N,N-diisopropylethylamine hydrochloride (DIPEA-CI) to attach a N,N-diisopropylethylamine (DIPEA) to chitosan to obtain a N,N-diisopropylethylamine-chitosan (DIPEA-Ch);
optionally reacting polyethylene glycol with N-Succinimidyl 3-(2-pyridyldithio) propionate (SPDP) to obtain PEG-SH and reacting said PEG-SH obtained with said DIPEA-Ch to obtain DIPEA-Ch-PEG.

13. The method of claim 12, further comprising the step of purifying said DIPEA-ch or said DIPEA-Ch-PEG and isolating said DIPEA-ch or said DIPEA-Ch-PEG.

14. The method of claim 13, wherein the step of purifying said DIPEA-ch or said DIPEA-Ch-PEG is effected by dialysis.

15-26. (canceled)
